(19) United States
(12) Patent Application Publication
(10) Pub. No.: US 2006/0142671 A1
(43) Pub. Date: Jun. 29, 2006

(54) Limb angle reading device for post-operative therapy

(76) Inventor: David Mark Solak, Seven Hills, OH (US)

Correspondence Address: DAVID MARK SOLAK, 4805 MAPLE HILL DRIVE, SEVEN HILLS, OH, 44131, US

(21) Appl. No.: 11/023,149

(22) Filed: Dec. 27, 2004

(51) Int. Cl. A61B 5/103

(52) U.S. Cl. 600/595

(57) ABSTRACT

A mechanism for viewing the angle of one's joint by simply attaching bands around the limbs relative to the affected joint. A read out may help a patient regain full range-of-motion after surgery, especially when ligaments must heal and joints are immobilized for a long time. An angle sensor attached to a hinge axis about the joint, sends a signal to a converter which converts that signal into a legible readout so the patient can record or make goals to regain motion in the affected joint. The bands are detachable so the patient can remove the mechanism after use.

### BACKGROUND OF INVENTION

1. Field of Invention

This invention is intended to display the angle of an upper limb in relation to the attached lower limb after an operation, or fractured. Many times, after an operation for instance, a joint needs its ligaments to be re-trained (stretched) to regain full motion. Using an osteotomy surgery on the leg as an example, the leg is immobile for many months until the bone heals. After the healing of the bone, it is necessary for the ligaments to be trained and stretched to allow full motion of the knee joint. A therapist will need to help a patient “re-stretch” the ligaments with very little angular motion of the limb at one time. It is a very painful process for the patient. This device is intended to give an angular readout of the progress a patient makes when re-training a limb.

2. Description of Prior Art

There is an angular knee support in prior art U.S. Pat. No. 5,403,350 issued Apr. 4, 1995 to McAtee that is intended for use with an MRI machine. The patent shows a patient on his/her stomach pulling the feet up with a cord. There is no indication of a reading of angle related to the limbs that are being lifted by the brace. This device is intended to be used in conjunction with a MRI machine. U.S. Pat. No. 5,810,729 issued Sep. 22, 1998 to the General Electric Company Medical Systems, is a method for measuring limb angle, but it requires an MRI machine. This system is not cost effective or mobile for the patient. There are many types of patented braces for limbs. It can be important to show angular progress of the joint. As of today, there is no indication of a mobile mechanism, besides a protractor, that indicates position of the joint.

### SUMMARY OF THE INVENTION

This mechanism will show an angular readout of a limb when therapy is needed to regain full motion of a limb, especially after an operation to repair damage or deformities. The readout can be viewed by just the patient or by multiple people of a medical staff. The device may help brace a joint, but most importantly give a visual reading in angular measurement to a patient and his/her therapist.

### BRIEF DESCRIPTION OF THE DRAWINGS

FIG. 1. Shows a leg with the mechanism attached and possible reading from the angle sensor

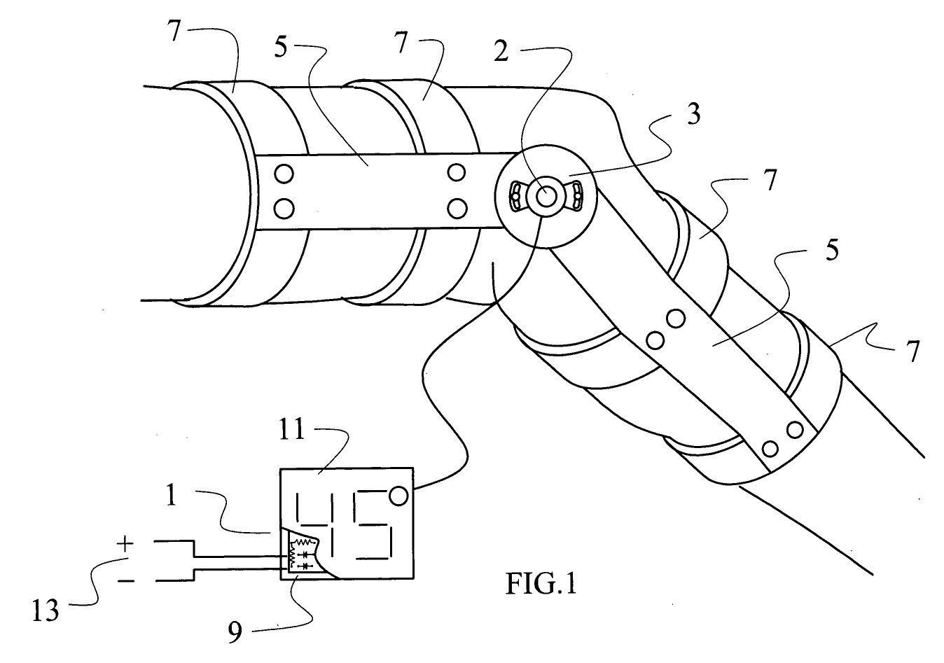

FIG. 2. Shows the opposite side of the leg with the mechanism attached.

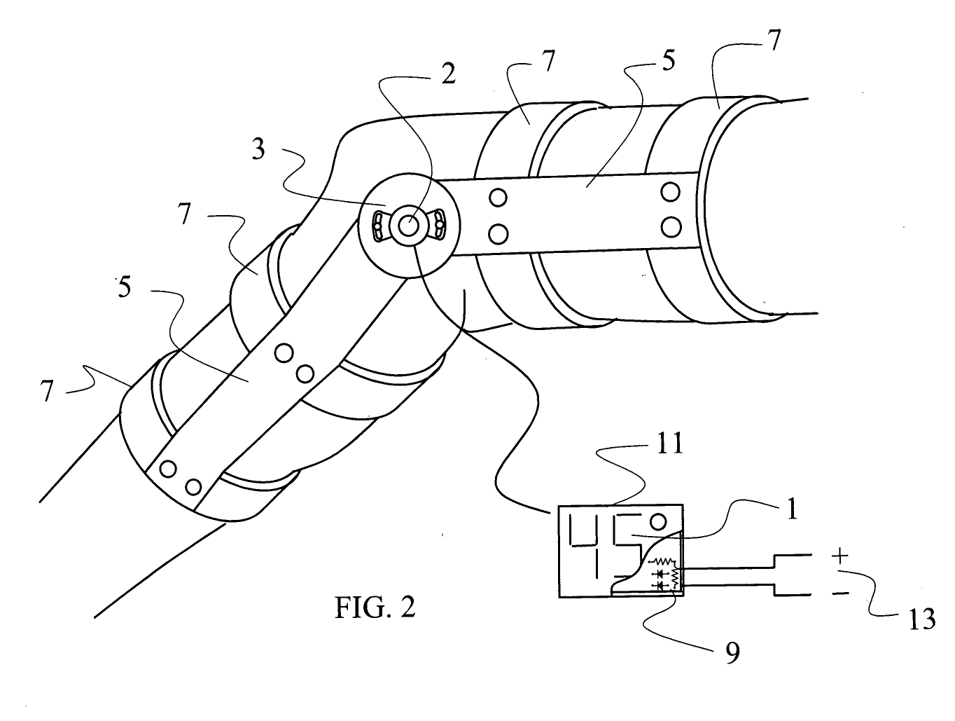

FIG. 3. Is a drawing of the mechanism as if looking from the hip outward.

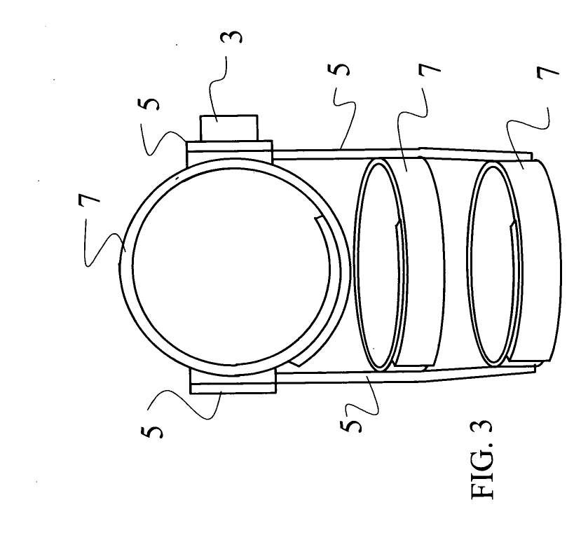

FIG. 4. Shows a block diagram describing precedence of the system.

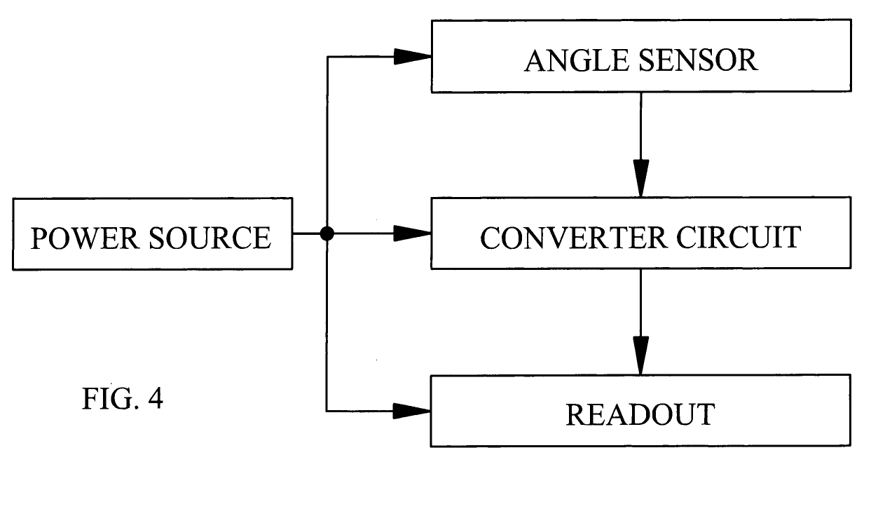

### DETAILED DESCRIPTION OF THE DRAWINGS

This mechanism will show a readout 1 in degrees relative to the position of a joint. An angle sensor 3 is positioned at the hinge 2 of two bars 5 near the joint axis. Four removable bands 7 secure the bars 5 and the angle sensor 3 to the relative limbs. A converter circuit 9 in readout box 11 will convert the signals from the angle sensor 3 into a legible readout 1. A power source 13 will produce the electricity to make the system operable.

I claim:

1. A device that attaches to a human limb comprising:
a. an electronic angle sensor to send electronic signals to a readout box; and
b. a converter circuit to convert the said electronic signals into a legible readout; and
c. removable bands to secure the device to the said relative limbs; and
d. bars to hold and position the said electronic angle sensor in place; and
e. a means to electrically power the said electronic angle sensor and said converter circuit.
